(19) United States
(12) Patent Application Publication
(10) Pub. No.: US 2014/0066808 A1
(43) Pub. Date: Mar. 6, 2014

(54) SIGNAL PROCESSING SYSTEM, SIGNAL PROCESSING APPARATUS, AND STORAGE MEDIUM

(71) Applicant: SONY CORPORATION, Tokyo (JP)

(72) Inventors: YOICHIRO SAKO, Tokyo (JP); KOHEI ASADA, Kanagawa (JP); TAKATOSHI NAKAMURA, Kanagawa (JP); AKIRA TANGE, Tokyo (JP); KAZUYUKI SAKODA, Chiba (JP); KATSUHISA ARATANI, Kanagawa (JP); MITSURU TAKEHARA, Tokyo (JP); KAZUHIRO WATANABE, Tokyo (JP); HIROYUKI HANAYA, Kanagawa (JP); YUKI KOGA, Tokyo (JP); TOMOYA ONUMA, Shizuoka (JP)

(73) Assignee: SONY CORPORATION, Tokyo (JP)

(21) Appl. No.: 13/968,717

(22) Filed: Aug. 16, 2013

(30) Foreign Application Priority Data
Aug. 29, 2012 (JP) 2012-188800

(51) Int. Cl. A61B 7/00

(52) U.S. Cl. CPC A61B 7/00
USPC 600/586

(57) ABSTRACT

There is provided a signal processing system including a first detection section which detects, from outside a body cavity, first audio signals of a prescribed part inside the body cavity, a second detection section which detects, from inside the body cavity, second audio signals of the prescribed part inside the body cavity, and a generation section which generates third audio signals based on the first audio signals and the second audio signals.

### BACKGROUND

The present disclosure relates to a signal processing system, a signal processing apparatus, and a storage medium.

While in the past stethoscopes have been used for listening to sounds within the body from outside of the body in order to examine such things as arrhythmia, heart murmurs and asthma, in recent years, endoscopes with a microphone attached have been proposed, which include mechanisms for detecting sounds at the distal end insertion portion or the like of the endoscope, and which detect sounds within the body along with an image of inside the body cavity.

Specifically, for example, JP 559-168832A discloses an endoscope apparatus which includes an optical fiber microphone mounted on an optical fiber, which guides light to the distal end insertion portion of an endoscope capable of imaging inside the body cavity. Further, JP H08-126603A discloses an endoscope apparatus which sends sound signals generated by a microphone, which is included at the distal end insertion portion of an endoscope, to a TV monitor, and outputs the sounds from the speakers of the TV monitor.

Further, JP 2001-104249A discloses an endoscope apparatus which is connected with a bone conduction type microphone capable of collecting sounds. Further, JP 2005-87297A discloses an endoscope apparatus which can accurately pick up vibrations of sound waves or the like inside the body cavity, by including a microphone at the distal end insertion portion.

Further, JP 2006-158515A discloses an endoscope apparatus which includes a microphone unit capable of attaching to and detaching from the distal end insertion portion of an endoscope.

### SUMMARY

Here, in all of the above described endoscope apparatuses, an imaging section which takes images of inside the body cavity is the main function, and a microphone which detects body sounds is subordinately included. Therefore, sound signals generated by the microphone are sent to an external apparatus (a television or the like which displays the captured image of inside the body cavity) as they are, and are simply output as sounds from the speakers of the external apparatus. Further, none of these disclose a combination of the body sounds collected inside the body cavity and the body sounds collected from outside the body cavity by a stethoscope.

However, since body sounds are important factors in the judgment of diseases, an apparatus has been sought after which records (stores) more accurate body sounds, and effectively processes the body sounds so that they can be used for diagnosis, in order to accurately understand the condition of a disease and for early detection or the like of an abnormal part (affected part).

Accordingly, the present disclosure proposes a signal processing system, a signal processing apparatus, and a storage medium capable of more effectively acquiring body sounds used for diagnosis.

According to an embodiment of the present disclosure, there is provided a signal processing system including a first detection section which detects, from outside a body cavity, first audio signals of a prescribed part inside the body cavity, a second detection section which detects, from inside the body cavity, second audio signals of the prescribed part inside the body cavity, and a generation section which generates third audio signals based on the first audio signals and the second audio signals.

Further, according to an embodiment of the present disclosure, there is provided a signal processing apparatus including a first detection section which detects, from outside a body cavity, first audio signals of a prescribed part inside the body cavity, and a generation section which generates third audio signals based on the first audio signals, and second audio signals of the prescribed part inside the body cavity detected by a second detection section which detects, from inside the body cavity, the second audio signals.

Further, according to an embodiment of the present disclosure, there is provided a non-transitory computer-readable storage medium having a program stored thereon, the program causing a computer to execute detecting, from outside a body cavity, first audio signals of a prescribed part inside the body cavity, detecting, from inside the body cavity, second audio signals of the prescribed part inside the body cavity, and generating third audio signals based on the first audio signals and the second audio signals.

Further, according to an embodiment of the present disclosure, there is provided a non-transitory computer-readable storage medium having a program stored thereon, the program causing a computer to execute detecting, from outside a body cavity, first audio signals of a prescribed part inside the body cavity, and generating third audio signals based on the first audio signals, and second audio signals of the prescribed part inside the body cavity detected from inside the body cavity.

According to the embodiments of the present disclosure described above, it becomes possible to more effectively acquire body sounds used for diagnosis.

### BRIEF DESCRIPTION OF THE DRAWINGS

FIG. 1 is an explanatory diagram for describing an outline of a sound collection system according to the embodiments of the present disclosure;

FIG. 2 is an explanatory diagram which shows a configuration of a capsule according to a first embodiment of the present disclosure;

FIG. 3 is an explanatory diagram which shows a configuration of inside and surrounding a signal processing section according to the first embodiment;

FIG. 4 is an explanatory diagram which shows a configuration of an auscultation apparatus according to the first embodiment;

FIG. 5 is an explanatory diagram which shows a configuration of inside and surrounding a signal processing section according to the first embodiment;

FIG. 6 is a sequence diagram which shows the operation processes of the sound collection system according to the first embodiment;

FIG. 7 is an explanatory diagram for describing a positional relation of the capsule, sound collection section, and target part according to the first embodiment;

FIG. 8 is an explanatory diagram for describing an example of a time difference correction process by a two way reference type noise reduction section according to the first embodiment;

FIG. 9 is an explanatory diagram for describing an example of a target sound generation process by a target sound extraction section according to the first embodiment; and

FIG. 10 is a sequence diagram which shows the operation processes of a sound collection system according to a second embodiment of the present disclosure.

### DETAILED DESCRIPTION OF THE EMBODIMENT(S)

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

The description will be given in the following order:

1. Outline of a Signal Processing System According to the Embodiments of the Present Disclosure

2. Each of the Embodiments

2-1. The first embodiment

(2-1-1) Configuration of the capsule

(2-1-2) Configuration of the auscultation apparatus

(2-1-3) Operations

(2-1-4) Effects

2-2. The second embodiment

(2-2-1) Operations

(2-2-2) Effects

3. Conclusion

### 1. OUTLINE OF A SIGNAL PROCESSING SYSTEM ACCORDING TO THE EMBODIMENTS OF THE PRESENT DISCLOSURE

The present disclosure can be implemented in various forms, such as in [2-1. The first embodiment] to [2-2. The second embodiment] described in detail as examples. Further, a sound collection system (signal processing system) according to each of the embodiments includes:

A. A first detection section (sound collection section 12) which detects, from outside a body cavity, first audio signals (body cavity external signals) of a prescribed part inside the body cavity;

B. A second detection section (capsule 2) which detects, from inside the body cavity, second audio signals (body cavity internal signals) of the prescribed part inside the body cavity; and

C. A generation section (signal processing section 130) which generates third audio signals (target signals), based on the first audio signals and the second audio signals.

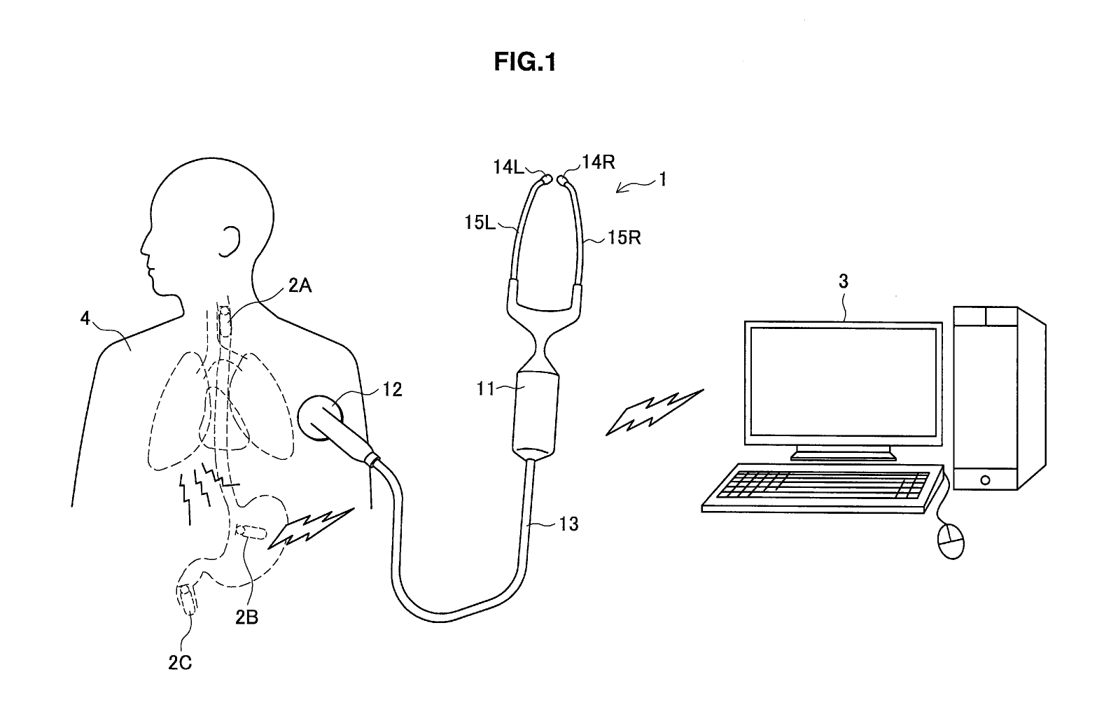

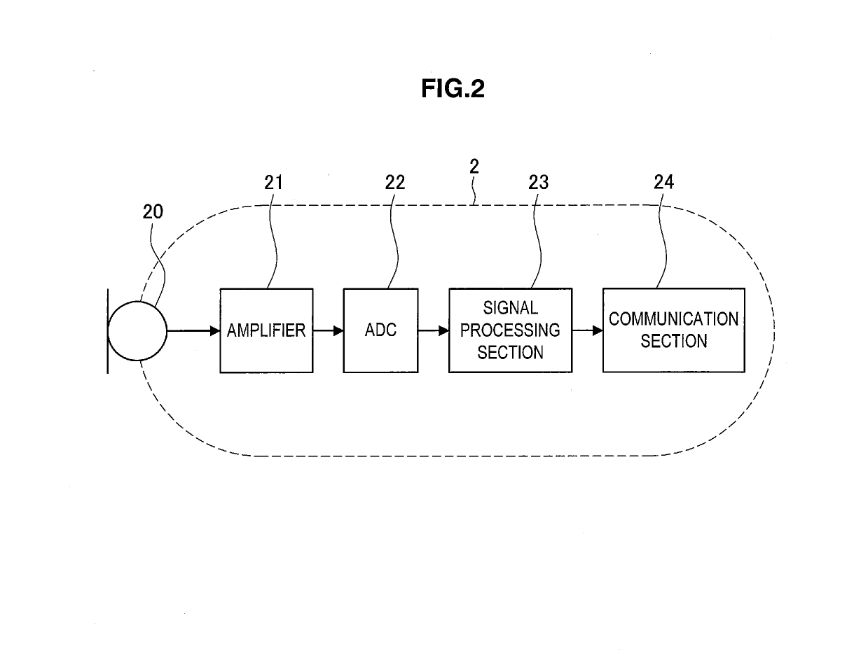

Hereinafter, first a basic configuration of such a sound collection system common to each of the embodiments of the present disclosure will be described with reference to FIGS. 1 and 2.

FIG. 1 is an explanatory diagram for describing an outline of a sound collection system according to the embodiments of the present disclosure. As shown in FIG. 1, the sound collection system includes an auscultation apparatus 1, a capsule type medical apparatus 2 (hereinafter, called the capsule 2) introduced into the body by being swallowed or the like by a test subject 4, and an external apparatus 3. Note that the sound collection system may include a plurality of the capsules 2 (2A, 2B, 2C). Further, the capsule 2 has a communication function, and is capable of performing transmission/reception of data with the auscultation apparatus 1 outside of the body. Also, the auscultation apparatus 1 has a similar communication function, and is capable of performing transmission/reception of data with the external apparatus 3.

Further, as shown in FIG. 1, the auscultation apparatus 1 includes a body section 11, a sound collection section 12, a cable 13, ear sections 14, and ear tube sections 15. The sound collection section 12 collects body sounds from outside the body cavity by contacting the body surface. Also, the cable 13 transfers the body sounds collected by the sound collection section 12 to the body section 11. On the other hand, the capsule 2 collects body sounds from inside the body cavity. Also, the capsule 2 transmits the body sounds collected from inside the body cavity to the body section 11, which has a communication function with the capsule 2.

The body section 11 generates sounds (target sounds) of a prescribed part (a target part) which is to be observed, by combining the body sounds collected from inside the body cavity by the capsule 2 and the body sounds collected from outside the body cavity by the sound collection section 12. The ear tube sections 15L and 15R transfer the target sounds generated by the body section 11 to the ear sections 14L and 14R. Also, the ear sections 14L and 14R output the target sounds transferred by the ear tube sections 15L and 15R.

Note that, in the present disclosure, body sounds are sounds generated within the body, and are, for example, heart sounds, respiratory sounds, a pulse, bowel sounds or the like. Further, the target part shows, for example, an internal organ such as the heart, lungs or digestive tract.

Further, the body section 11 transmits the generated target sounds to the external apparatus 3, and may record the target sounds to the external apparatus 3. Further, the auscultation apparatus 1 can receive and reproduce previous target sounds accumulated in the external apparatus 3.

Hereinafter, such a sound collection system according to the embodiments of the present disclosure will be described in detail by including a plurality of embodiments. Note that, in each of the embodiments described hereinafter, an electronic auscultation apparatus will be used as an example of a signal processing apparatus which has the first detection section (sound collection section 12) according to the embodiments of the present disclosure. Further, while a capsule type medical apparatus is used as an example of the second detection section according to the embodiments of the present disclosure, the present disclosure is not limited to such an example. For example, the second detection section may be an endoscope type medical apparatus. Further, as shown in FIG. 1, while a PC (Personal Computer) is shown as an example of the external apparatus 3 according to the embodiments of the present disclosure, the present disclosure is not limited to such an example. For example, the external apparatus 3 may be a server, a smart phone, a PDA (Personal Digital Assistant), a notebook PC, a mobile phone, a portable music player, a mobile video processing apparatus, a portable game machine or the like.

### 2. EACH OF THE EMBODIMENTS

### 2-1. The First Embodiment

According to the present embodiment, it is possible to more clearly generate target sounds originating at a target part inside the body cavity, by combining body sounds collected from inside the body cavity by the capsule 2 and body sounds collected from outside the body cavity by the auscultation apparatus 1. Hereinafter, a configuration of the capsule 2 and the auscultation apparatus 1 included in the sound collection system according to the present embodiment will be sequentially described with reference to FIGS. 2 to 5.

[2-1-1. Configuration of the Capsule 2]

The capsule 2, as described above, collects body sounds from inside the body cavity of a test subject 4, and transmits the collected body sounds to the auscultation apparatus 1. In this case, the capsule 2 may transmit the collected body sounds upon performing prescribed signal processes for the collected body sounds. For example, in the case where a prescribed part or a target part such as an internal organ is specified as an observation target of the body sounds, sounds originating from other parts or internal organs may become unnecessary noise. Accordingly, in order to improve the S/N ratio (signal/noise ratio) by decreasing the unnecessary noise, the capsule 2 may perform band restriction or the like as a signal process for the collected body sounds. A configuration of the capsule 2 which performs a signal process such as band restriction or the like will be described with reference to FIG. 2.

FIG. 2 is an explanatory diagram which shows a configuration of the capsule 2 according to a first embodiment of the present disclosure. As shown in FIG. 2, the capsule 2 has a sound collection section 20, an amplifier 21, an ADC (Analog Digital Convertor) 22, a signal processing section 23, and a communication section 24.

(Sound Collection Section)

The sound collection section 20 has a function of a microphone. The sound collection section 20 is a detection section which collects (detects) body sounds from inside the body cavity, and outputs the collected body sounds as audio signals. Further, the sound collection section 20 has directivity, and may collect only sounds from a target part direction. Note that, hereinafter, the audio signals detected by the sound collection section 20 will be called body cavity internal signals.

(Amplifier)

The amplifier 21 has a function which amplifies audio signals. The amplifier 21 amplifies and outputs the body cavity internal signals output from the sound collection section 20.

(ADC)

The ADC 22 is an electronic circuit which converts analog electrical signals into digital electrical signals. The ADC 22 converts the body cavity internal signals output from the amplifier 21 from analog signals into digital signals, and outputs the digital signals.

(Signal Processing Section)

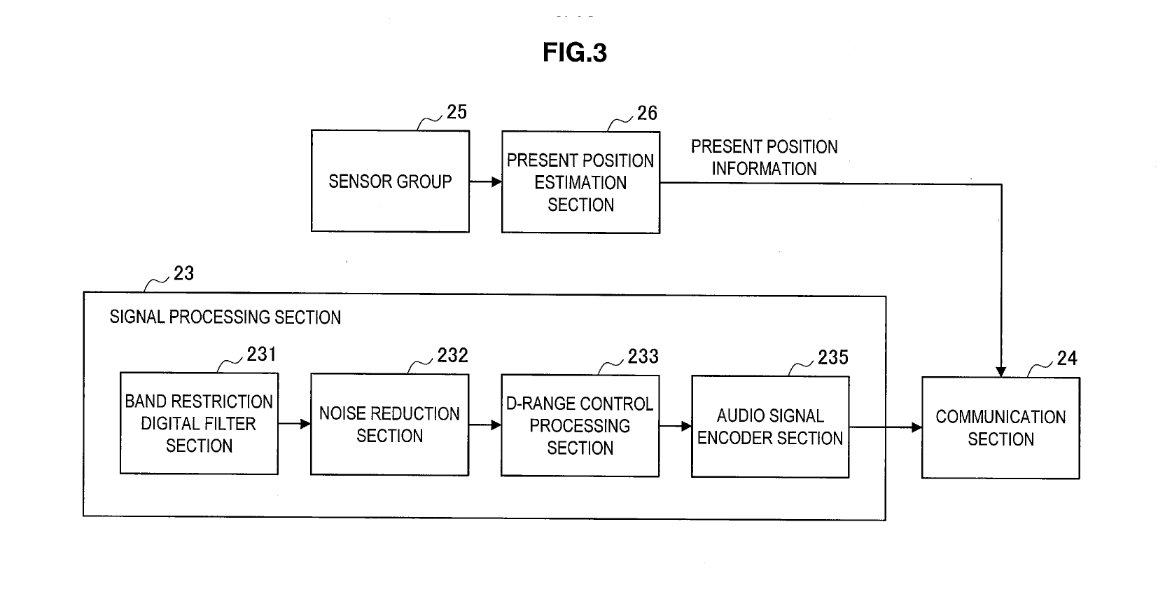

The signal processing section 23 has a function which performs prescribed signal processes for the audio signals. The signal processing section 23 performs prescribed signal processes for the body cavity internal signals output from the ADC 22, and outputs the body cavity internal signals. The signal processing section 23 may be implemented, for example, by an operation apparatus such as a DSP (Digital Signal Processor), or an MPU (Micro-Processing Unit). Here, a specific configuration of the signal processing section 23 will be described in detail with reference to FIG. 3.

FIG. 3 is an explanatory diagram which shows a configuration of inside and surrounding the signal processing section 23 according to the first embodiment. As shown in FIG. 3, the signal processing section 23 includes a band restriction digital filter section 231, a noise reduction section 232, a D-range control processing section 233, and an audio signal encoder section 235. Further, the capsule 2 additionally includes a sensor group 25 and a present position estimation section 26.

(Sensor Group)

The sensor group 25 is constituted, for example, by pressure sensors, tactile sensors, imaging sensors, acceleration sensors, pH sensors or the like, and detects a state of the body cavity interior surrounding the capsule 2.

(Present Position Estimation Section)

The present position estimation section 26 estimates which part, or within which internal organ, the capsule 2 is presently at, based on each sensor value (pH value or the like) detected by the sensor group 25, and outputs an estimation result as present position information. Note that, while in the present disclosure the position is described as indicating an absolute position, it may be described as a relative position from a prescribed part of the test subject 4, a prescribed apparatus, or the like.

(Communication Section)

The communication section 24 has a function which transmits/receives data wirelessly. For example, the communication section 24 wirelessly transmits the body cavity internal signals output from the signal processing section 23, the present position information output from the present position estimation section 26, the sensor values output by the sensor group 25, and the like. Further, the communication section 24 collects sounds from the sound collection section 20, and may wirelessly transmit the body cavity internal signals, the present position information, the sensor values, and the like via the amplifier 21 and the ADC 22. Note that the communication system of the communication section 24 is not particularly limited, and may be, for example, WiFi, Bluetooth (registered trademark), ZigBee or the like.

(Band Restriction Digital Filter Section)

The band restriction digital filter section 231 (Hereinafter, called the band restriction DF section 231) has a function to reduce or let through the audio signals of prescribed frequency bands. The band restriction DF section 231 can improve the S/N ratio, by letting through the prescribed frequency bands from among the body cavity internal signals output from the ADC 22. Further, the band restriction DF section 231 may be digitalized by a BPF (Band-Pass Filter), an LPF (Low-Pass Filter), an HPF (High-Pass Filter) or the like, and it becomes possible for high precision control of a steep filter, a direct phase filter or the like.

(Noise Reduction Section)

The noise reduction section 232 (hereinafter, called the NR section 232) has a function which cuts a prescribed noise component. The NR section 232 cuts a prescribed noise component from the body cavity internal signals output from the band restriction DF section 231. In the present embodiment, in the case where a continuous and regular sound, such as a blood flow sound, is contained within the detected body cavity internal signals at the time when, for example, an observation to be focused on is a heart sound, the NR section 232 treats this blood flow sound as noise, and it is possible to cut the blood flow sound. Specifically, the NR section 232 may estimate a noise (here, the blood flow sound), based on the results of a sound collection recording and a frequency analysis of a certain period, and may use a technique such as SS (Spectrum Subtraction), which subtracts this noise from the audio signals within an observation period on a frequency axis.

(D-Range Control Processing Section)

The D-range control processing section 233 has a function which controls the width of the volume of the audio signals. The D-range control processing section 233 reduces the load of the processing resources for the encoder section 235, which is described later, and reduces the transmission capacity by the communication section 24, by controlling the width of the volume of the body cavity internal signals output from the NR section 232. As a result, since the circuit size and the power consumption of the capsule 2 are reduced, the capsule 2 is further reduced in size, and the burden of the test subject 4 who swallows the capsule 2 will also be reduced.

(Audio Signal Encoder Section)

The audio signal encoder section 235 (hereinafter, called the encoder section 235) has a function which encodes audio signals. The encoder section 235 encodes the body cavity internal signals output from the D-range control processing section 233, and outputs the encoded body cavity internal signals. The coding system is not particularly limited, and may be, for example, MP3 (MPEG Audio Layer-3), AAC (Advanced Audio Coding) or the like. Further, the coding system of the encoder section 235 may be a suitable coding system corresponding to the communication system of the communication section 24.

Note that the capsule 2, as described above, performs sound collection and signal processes, based on information which specifies the target part to be observed, position information of the target part, and information (parameters) such as setting information which sets the signal processes by the signal processing section 23. Such parameters may be set, for example, prior to the capsule 2 being introduced into the body of the test subject 4. Also, the parameters may be set via wireless communication from the outside after the capsule 2 has been introduced into the body of the test subject 4.

[2-1-2. Configuration of the Auscultation Apparatus 1]

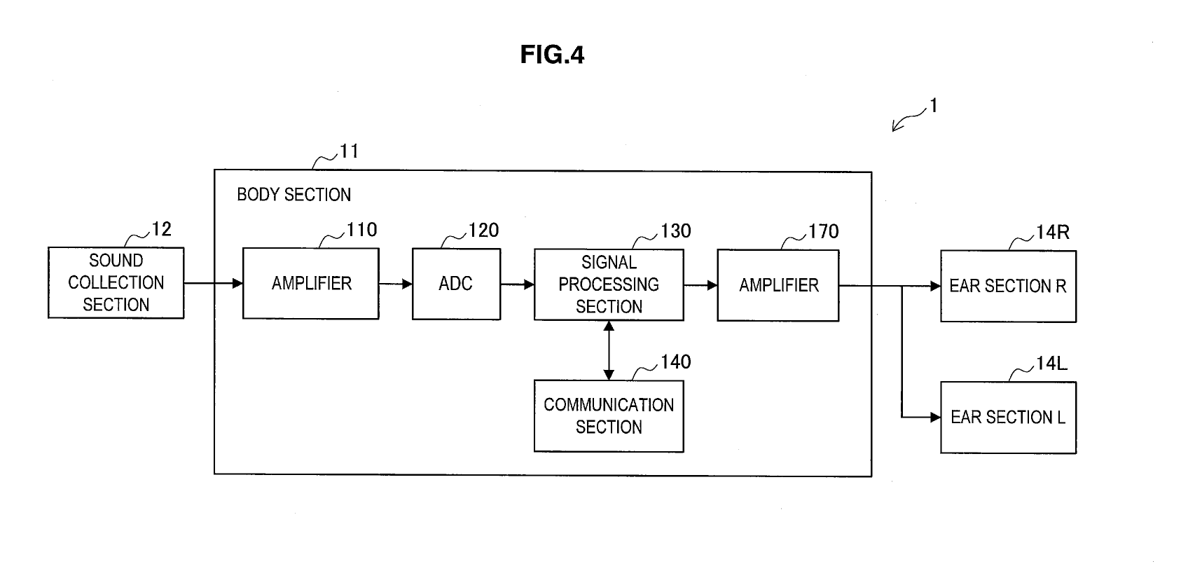

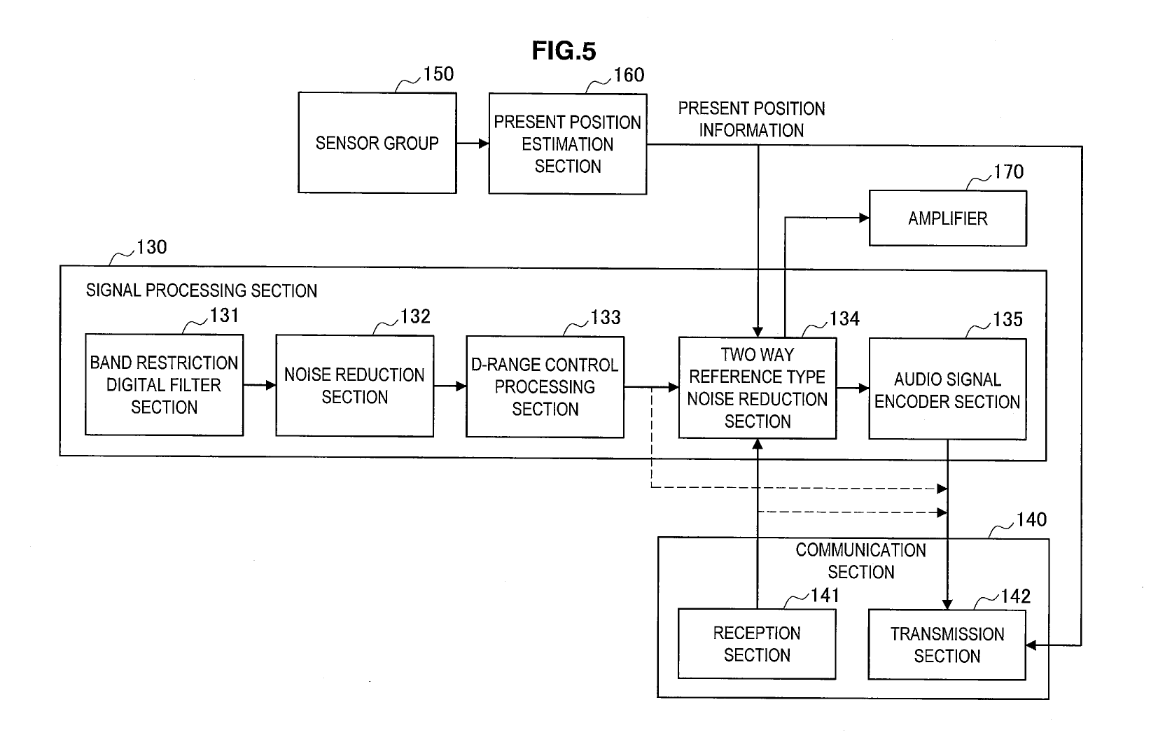

Heretofore, a configuration of the capsule 2 has been described. To continue, a configuration of the auscultation apparatus 1 will be described with reference to FIGS. 4 and 5.

FIG. 4 is an explanatory diagram which shows a configuration of the auscultation apparatus 1 according to the first embodiment. As shown in FIG. 4, the body section 11 of the auscultation apparatus 1 has an amplifier 110, an ADC 120, a signal processing section 130, a communication section 140, and an amplifier 170.

(Amplifiers and ADC)

The amplifier 110 and the ADC 120 have functions similar to those of the amplifier 21 and the ADC 22, and perform amplification of the audio signals output from the sound collection section 12 (hereinafter, called body cavity external signals), conversion of the body cavity external signals into digital signals, and output of the digital signals. The amplifier 170 has a function similar to that of the amplifier 21, and amplifies and outputs the body cavity external signals and target signals output from the signal processing section 130, which is described later.

(Signal Processing Section)

The signal processing section 130 generates target signals (signals of target sounds), by combining the body cavity internal signals detected by the capsule 2 (signals of body sounds collected from inside the body cavity) and the body cavity external signals detected by the sound collection section 12 (signals of body sounds collected from outside the body cavity). The signal processing section 130, similar to that of the signal processing section 23, may be implemented, for example, by an operation apparatus such as a DSP (Digital Signal Processor), or an MPU (Micro-Processing Unit). Here, a specific configuration of the signal processing section 130 will be described in detail with reference to FIG. 5.

FIG. 5 is an explanatory diagram which shows a configuration of inside and surrounding the signal processing section 130 according to the first embodiment. As shown in FIG. 5, the signal processing section 130 includes a band restriction digital filter section 131, a noise reduction section 132, a D-range control processing section 133, a two way reference type noise reduction section 134, and an encoder section 135. Further, the body section 11 additionally includes a sensor group 150 and a present position estimation section 160.

(Sensor Group)

The sensor group 150 is constituted, for example, by tactile sensors, acceleration sensors, gyro sensors or the like included in the sound collection section 12, and detects a state surrounding the sound collection section 12. Further, the sensor group 150 may be constituted by cameras, infrared sensors, depth sensors or the like included in the body section 11, and may detect from a bird's-eye view a state surrounding the sound collection section 12.

(Present Position Estimation Section)

The present position estimation section 160 estimates which position of the surface the sound collection section 12 is presently contacting, based on each sensor value (a detection result or the like by the infrared sensors and tactile sensors) detected by the sensor group 150, and outputs an estimation result as present position information.

(Communication Section)

The communication section 140 has a function similar to that of the communication section 24. In more detail, the communication section 140 has a function of a reception section 141 which receives data and a function of a transmission section 142 which transmits data.

(Reception Section)

The reception section 141 outputs the body cavity internal signals and present position information of the capsule 2 received from the capsule 2 to the two way reference type noise reduction section 134. Further, the reception section 141 may output previous body cavity external signals, body cavity internal signals and target signals received from the external apparatus 3 to the two way reference type NR section 134. Note that while the present embodiment shows an example in which the reception section 141 is arranged in the body section 11 along with the transmission section 142 as an example, the present disclosure is not limited to such an example. For example, the reception section 141 may be arranged in the sound collection section 12 in a state separated from the transmission section 142. In this case, the reception section 141 can receive data from the capsule 2 at a position closer to the capsule 2.

(Transmission Section)

The transmission section 142 wirelessly transmits, to the external apparatus 3, the target signals output from the encoder section 135, which is described later, and the present position information of the sound collection section 12 output from the present position estimation section 160. Further, the transmission section 142 may wirelessly transmit, to the external apparatus 3, the body cavity external signals output by the D-range control processing section 133, which is described later, and the body cavity internal signals and present position information of the capsule 2 output from the reception section 141. Further, the transmission section 142 may collect sounds from the sound collection section 12, and may transmit, to the external apparatus 3, the body cavity external signals and the present position information of the sound collection section 12 via the amplifier 110 and the ADC 120. In addition, the transmission section 142 may transmit, to the capsule 2, parameters used for sound collection and signal processes in the above described capsule 2. Note that, while the present embodiment describes an example in which the communication section 140 wirelessly communicates with the external apparatus 3, it may also be by wired communication.

(Band Restriction Digital Filter Section)

The band restriction digital filter section 131 (Hereinafter, called the band restriction DF section 131) has a function similar to that of the band restriction DF section 231. The noise reduction section 132 (hereinafter, called the NR section 132) has a function similar to that of the NR section 232. The D-range control processing section 133 has a function similar to that of the D-range control processing section 233. Also, the band restriction DF section 131, the NR section 132, and the D-range control processing section 133 perform signal processes similar to those of the band restriction DF section 231, the NR section 232, and the D-range control processing section 233, for the body cavity external signals output by the sound collection section 12.

(Two Way Reference Type Noise Reduction Section)

The two way reference type noise reduction section 134 (hereinafter, called the two way reference type NR section 134) has a function which extracts audio signals by improving the S/N ratio of audio signals from a prescribed sound source using a plurality of audio signals. In the present embodiment, the two way reference type NR section 134 generates and outputs target signals, by combining the body cavity internal signals received from the reception section 141 and the body cavity external signals output from the band restriction DF section 131. Note that, when combining the body cavity internal signals and the body cavity external signals, the two way reference type NR section 134 uses the present position information of the sound collection section 12 output from the present position estimation section 160 and the present position information of the capsule 2 output from the reception section 141.

(Audio Signal Encoder Section)

The audio signal encoder section 135 (hereinafter, called the encoder section 135) has a function similar to that of the encoder section 235, and encodes the audio signals output from the two way reference type NR section 134, and outputs the encoded audio signals.

Heretofore, each constituent element of the body section 11 of the auscultation apparatus 1 has been described in detail. Note that, other than that described above, the body section 11 may include a storage section which stores at least one of the body cavity external signals, the body cavity internal signals and the target signals, an operation input section which performs various settings, a battery which accumulates operation power of the auscultation apparatus 1, and the like.

Further, the auscultation apparatus 1 performs sound collection and signal processes, based on information which specifies the target part to be observed, and information (parameters) such as position information of the target part. Such parameters used by the auscultation apparatus 1 and the capsule 2 may be set by the operation input section. Also, the target part may be an internal organ at a distance closest to the position of the sound collection section 12, or positioned in front of the sound collection section 12, and may be automatically set by the auscultation apparatus 1 in accordance with the position of the sound collection section 12.

Further, the auscultation apparatus 1 may store the body cavity external signals, the body cavity internal signals and the target signals when they are acquired, and may transmit these signals to the external apparatus 3 at a subsequent given timing.

Further, the storage section may be constituted, for example, by a semiconductor memory, a magnetic disk, an optical disk or the like, and may store various data which may be necessary for the auscultation apparatus 1 to function. Also, the auscultation apparatus 1 may operate by reading and executing programs stored in the storage section.

[2-1-3. Operations]

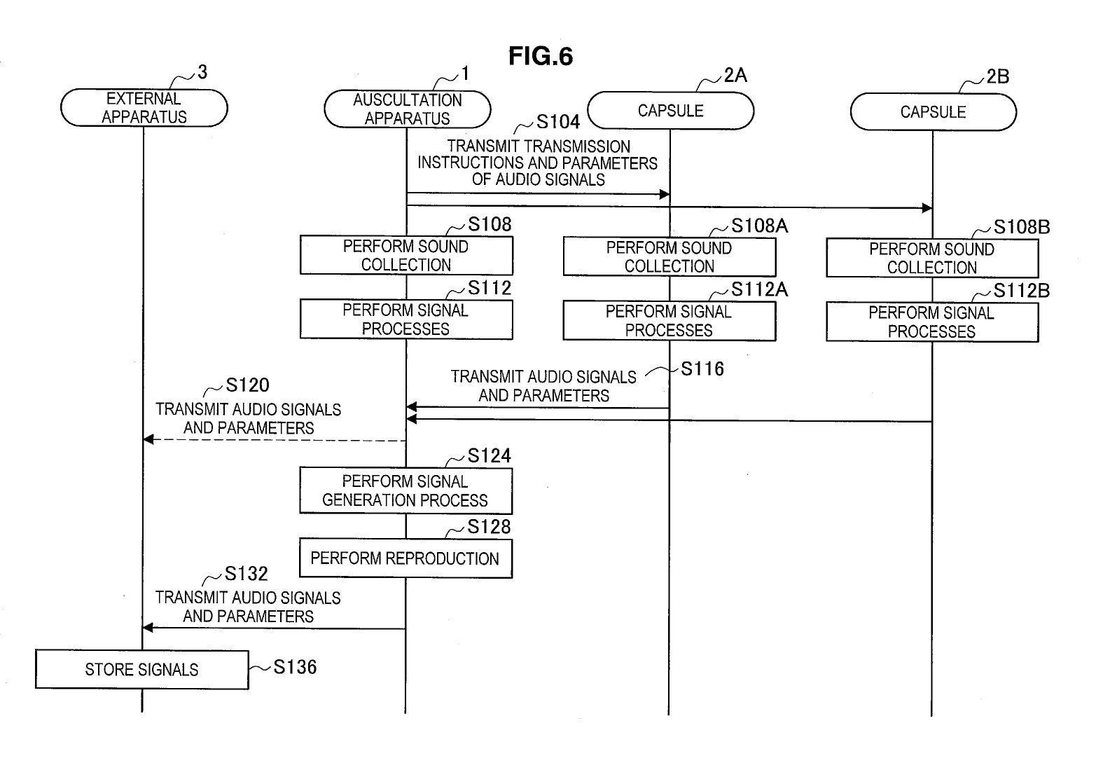

Heretofore, a configuration of the sound collection system according to the present embodiment has been described. To continue, the operations of the sound collection system according to the present embodiment will be described with reference to FIGS. 6 to 9.

FIG. 6 is a sequence diagram which shows the operation processes of the sound collection system according to the first embodiment. As shown in FIG. 6, the auscultation apparatus 1 can reproduce target sounds by using body sounds collected by one or more of the capsules 2.

First, in step S104, the transmission section 142 transmits transmission instructions and parameters of the audio signals to the capsules 2 introduced into the body cavity, and these are received by the communication section 24. The parameters here are parameters used for sound collection and signal processes in the above described capsules 2.

Note that the transmission section 142 may transmit transmission instructions to a capsule 2, from among the one or more capsules 2 introduced into the body cavity, which is located at a position closest to the sound collection section 12 positioned outside of the body. Also, the transmission section 142 may transmit transmission instructions to capsules 2 which are located within a range of a prescribed distance from the position of the sound collection section 12. Hereinafter, an example will be described in which the transmission section 142 transmits transmission instructions to capsules 2A and 2B.

To continue, in step S108, the sound collection section 12 collects body sounds from outside the body cavity. Then, in step S112, the signal processing section 130 performs signal processes for the body sounds collected by the sound collection section 12. Specifically, the signal processing section 130 performs a band restriction by the band restriction DF section 131, performs cutting of a noise component by the NR section 132, and performs control of the width of the volume by the D-range control processing section 133, for the body cavity external signals output by the sound collection section 12.

Then, the capsule 2A, which has received transmission instructions from the auscultation apparatus 1, sets the target part shown by the parameters attached to the transmission instructions as a target, and performs sound collection and signal processes of the body sounds. In more detail, in step S108A, the sound collection section 20 collects body sounds from inside the body cavity. Then, in step S112A, the signal processing section 23 performs signal processes for the body sounds collected by the sound collection section 20. Specifically, the signal processing section 23 performs a band restriction by the band restriction DF section 231, performs cutting of a noise component by the NR section 232, and performs control of the width of the volume by the D-range control processing section 233, for the body cavity internal signals output by the sound collection section 20.

Since the operations in steps S108B and S112B by the capsule 2B are similar to those of steps S108A and S112A described above for the capsule 2A, a description of them will be omitted here.

Afterwards, in step S116, the capsules 2A and 2B transmit the body cavity internal signals and parameters to the auscultation apparatus 1, and these are received by the reception section 141. The parameters here are present position information of the capsules 2, sensor values which show a state surrounding the capsules 2, and the time when the sound collection section 20 collected the sounds.

To continue, in step S120, the auscultation apparatus 1 transits, to the external apparatus 3, the body cavity external signals output by the D-range control processing section 133 in step S112, and the body cavity internal signals and parameters received by the reception section 141 in step S116. The parameters here are present position information of the sound collection section 12 and the capsules 2, and the time when the sound collection section 12 and the sound collection section 20 collected the sounds.

Then, in step S124, the auscultation apparatus 1 performs signal generation processes. In detail, the two way reference type NR section 134 performs a time difference correction process and a target sound generation process. The time difference correction process is a process in which the two way reference type NR section 134 corrects and synchronizes a time difference between the body cavity external signals output by the D-range control processing section 133 in step S112, and the body cavity internal signals received by the reception section 141 in step S116. Further, the target sound generation process is a process which separates the target signals from the body cavity external signals and the body cavity internal signals synchronized by the time difference correction process. Note that the time difference correction process and the target sound generation process will be described in detail later on.

Afterwards, in step S128, the auscultation apparatus 1 reproduces the target sounds based on the target signals generated by the two way reference type NR section 134. In more detail, the amplifier 170 amplifies the target signals output from the two way reference type NR section 134, and the ear sections 14R and 14L reproduce the target signals amplified by the amplifier 170 as target sounds.

Next, in step S132, the auscultation apparatus 1 transmits the target signals and parameters to the external apparatus 3. In more detail, the encoder section 135 encodes the target signals output from the two way reference type NR section 134, and the transmission section 142 transmits the target signals encoded by the encoder section 135 to the external apparatus 3. The parameters here are present position information of the sound collection section 12 and the capsules 2, and the time when the sound collection section 12 and the sound collection section 20 collected the sounds. Note that step S132 may be executed prior to step S128.

Finally, in step S136, the external apparatus 3 stores the target signals and parameters received from the auscultation apparatus 1 in steps S120 and S132.

(Time Difference Correction Process)

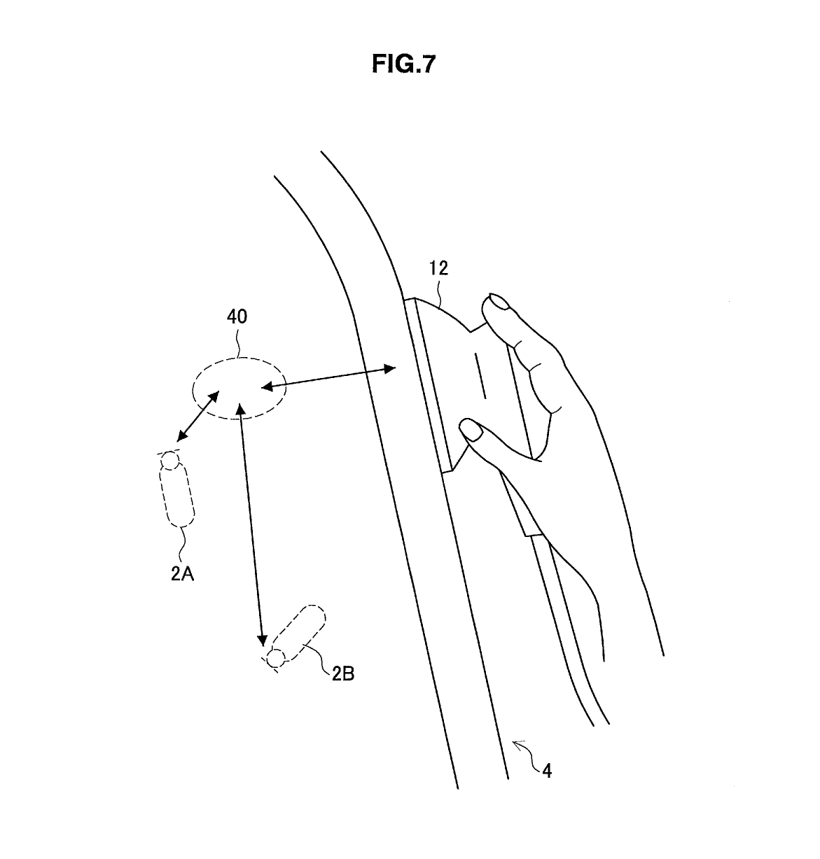

Hereinafter, the time difference correction process by the two way reference type NR section 134 in step S124 will be described in detail. First, the cause of a time difference occurring between the body sounds collected by the sound collection section 12 and the body sounds collected by the capsules 2 will be described with reference to FIG. 7.

FIG. 7 is an explanatory diagram for describing a positional relation of the capsules 2, the sound collection section 12, and the target part according to the first embodiment. As shown in FIG. 7, the distance up to the target part 40 can be different for each of the sound collection section 12 and the capsules 2A and 2B. Further, configurations can be present in which muscles, body fluids, internal organs, bones, skin or the like are different between the target part 40 and the auscultation apparatus 1 and capsules 2A and 2B.

Generally, the conductibility of sound changes in accordance with the distance up to the sound source and the material of substances or the like present in between the sound source. Therefore, a time difference is generated corresponding to the distance up to the target part 40 and the materials or the like, for the body sounds collected by the sound collection section 12, the body sounds collected by the capsule 2A, and the body sounds collected by the capsule 2B. Accordingly, the two way reference type NR section 134 corrects and synchronizes the time difference of the sounds in accordance with the distance up to the target part 40 and the materials or the like. Here, the two way reference type NR section 134 estimates the distance up to the target part (40) by absolute position information, and estimates the materials by sensor values which show a state surrounding the capsules 2.

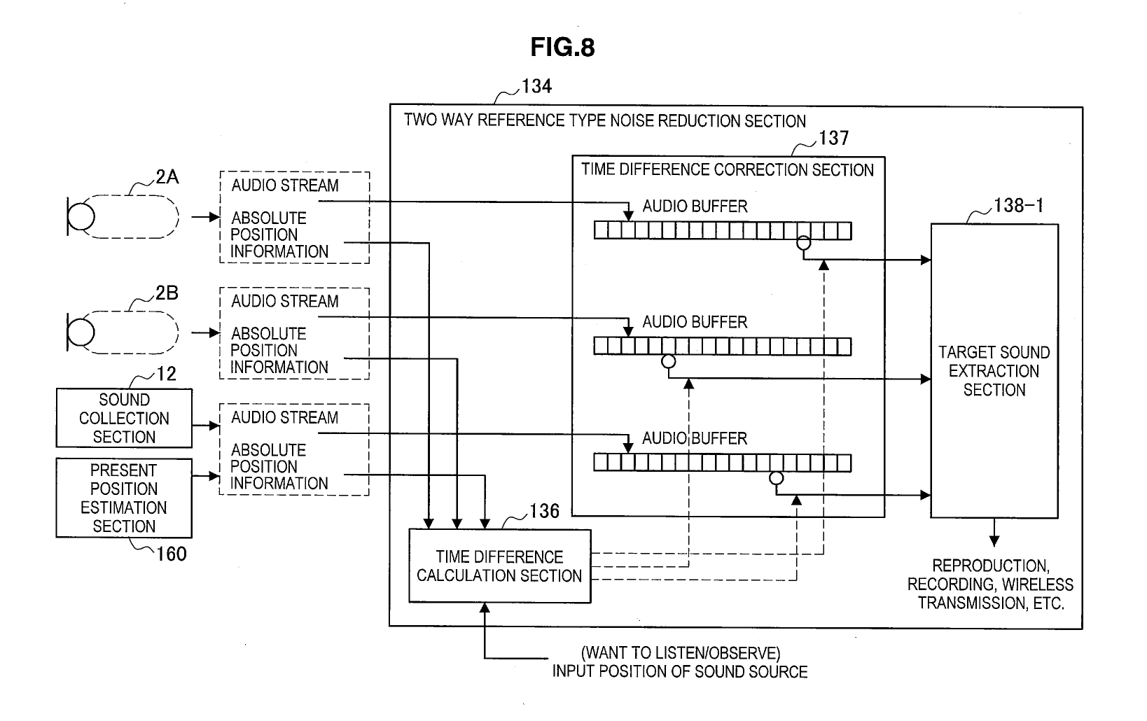

A specific example of such a time difference correction process will be described hereinafter with reference to FIG. 8.

FIG. 8 is an explanatory diagram for describing an example of a time difference correction process by the two way reference type NR section 134 according to the first embodiment. As shown in FIG. 8, the two way reference type NR section 134 has a function of a time difference calculation section 136, a function of a time difference correction section 137, and a function of a target sound extraction section 138.

The time difference calculation section 136 calculates a time difference between each audio stream, based on the present position information of the sound collection section 12 output by the present position estimation section 160, the present position information of the capsules 2A and 2B received from the capsules 2A and 2B, and the position information of the target part. The time difference correction section 137 first buffers the audio stream of the body sounds collected by the capsules 2A and 2B. Then, the time difference correction section 137 reads each audio stream from the stream position corresponding to the time difference calculated by the time difference calculation section 136, and outputs each audio stream to the target sound extraction section 138.

Note that, while in the above description the time difference calculation section 136 buffers the audio streams and then afterwards reads each audio stream from the stream position corresponding to the time difference, the present disclosure is not limited to such an example. For example, the time difference calculation section 136 may write each audio stream to the stream position corresponding to the time difference during buffering.

In this way, the time difference calculation section 136 and the time difference correction section 137 correct and synchronize a time difference between the body cavity external signals and the body cavity internal signals. To continue, the target sound extraction section 138 extracts and generates the target sounds.

(Target Sound Generation Process)

Hereinafter, the target sound generation process by the two way reference type NR section 134 in step S124 will be described in detail by using a specific example.

For example, a body cavity external signal T by the auscultation apparatus 1, a body cavity internal signal C₁ by the capsule 2A, and a body cavity internal signal C₂ by the capsule 2B are represented, such as shown below, as signals in which a heart sound X, a respiratory sound Y, and a noise N which are sounds not able to be classified as either of these, are mixed.

Body cavity external signal T by the auscultation apparatus 1=A₀*X+B₀*Y+N₀ (Equation 1)

Body cavity internal signal C₁ by the capsule 2A=A₁*X+B₁*Y+N₁ (Equation 2)

Body cavity internal signal C₂ by the capsule 2B=A₂*X+B₂*Y+N₂ (Equation 3)

Here, A(A₀, A₁, A₂) and B(B₀, B₁, B₂) are treated as coefficients.

Note that the coefficients A and B may be perceived as transfer functions of each sound collection section from the sound source. When describing coefficient A in more detail, coefficients A₀, A₁ and A₂ may be perceived as transfer functions directed from the heart position to the positions of the sound collection section 12 and the capsules 2A and 2B, respectively. The coefficients A₀, A₁ and A₂ show, by frequency characteristics, the conductivity of the sounds from the heart position up to the position of each sound collection section, and are determined by the position of each sound collection section and the materials of the transfer route.

Specifically, the coefficients A₀, A₁ and A₂ estimate the conduction characteristics of the sounds from the heart to each position, in accordance with experimental rules or simulations. Also, estimation may be performed by the sensor values detected by the sensor group 25. Further, in the case where a test subject 4 has an abdominal incision for surgery, transfer functions are measured between parts which can become target parts such as the heart or lungs, and parts in which the capsule 2 can pass through such as the stomach tube or the digestive tract, and these transfer functions may be used afterwards as the coefficient A.

Further, more simply, the coefficients A₀, A₁ and A₂ are determined by the distance from the heart position up to the position of each sound collection section, and by a temporal delay due to an attenuation degree of the sounds and the distances, even if the conductivity of the sounds of the transfer route are not yet known.

Since the coefficient B is similar to that of the above described coefficient A, a description of it will be omitted here.

Further, for example, upon specifying the part which becomes a noise generation source, N₀, N₁ and N₂ may be set to the body sounds previously collected at the time when the capsule 2 passed through the vicinity of the noise generation source.

As described above, in the case where A, B and N are already known, or can be calculated by different operations, the target sound extraction section 138 can calculate X and Y, by requesting an inverse matrix of the above described equations 1-3, or by solving simultaneous equations or the like.

On the other hand, the two way reference type NR section 134 can calculate X and Y, with A, B and N not yet known, by various techniques of sound source separation, such as blind sound source separation, blind signal source separation, main component analysis, or independent component analysis, or by a combination of these techniques.

(A Simpler Target Sound Generation Process)

Further, there are cases where the target sound extraction section 138 can extract target sounds more simply, when compared to the above described target sound generation process. First, in order to simplify the description, the body cavity external signal T by the auscultation apparatus 1 and the body cavity internal signal C₁ by the capsule 2 are represented, such as shown below, as signals in which the heart sound X and the noise N are mixed.

Body cavity external signal T by the auscultation apparatus 1=A₀*X+N₀ (Equation 4)

Body cavity internal signal C₁ by the capsule 2A=A₁*X+N₁ (Equation 5)

In this case, in the case where the frequency distribution characteristics of the noise N is obviously different to that of the heart sound X, the target sound extraction section 138 can extract the heart sound X with a good S/N ratio, by requesting α so that the frequency distribution characteristics of the noise N are canceled out from the body cavity external signal T at the time of setting (T−αC₁). Such an α can minimize (N₀−αN₁) for {(A₀−αA₁)X+(N₀−αN₁)}.

Note that, in the case where the heart sound X is a low frequency region only, since the time delay elements in the transfer functions can be disregarded, A₀ and A₁ can be perceived as gain values which show respective volumes, and (A₀-αA₁) can be perceived as a scalar value. Here, since a waveform shape, a time transition and the like are important as body sounds used for diagnosis, the target sound extraction section 138 may not calculate (A₀−αA₁).

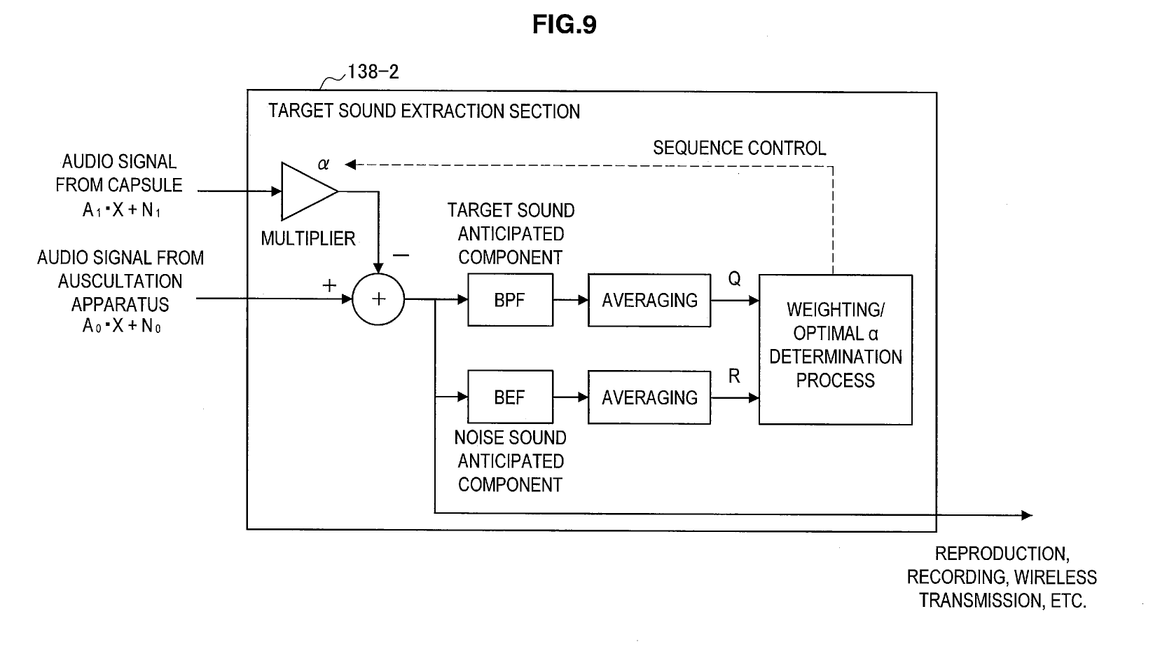

A specific example which requests such an α will be described hereinafter with reference to FIG. 9.

FIG. 9 is an explanatory diagram for describing an example of a target sound generation process by the target sound extraction section 138 according to the first embodiment. As shown in FIG. 9, first (T−αC₁) is calculated by using a prescribed α. Then, an optimal α, which can extract the heart sound X with a good S/N ratio, is searched for while α is successively changed.

In the case where the frequency distribution characteristics of the heart sound X are already known, the target sound extraction section 138 calculates a target sound anticipated component by a BPF (Band-Pass Filter) so that a band which is central to the heart sound X is let through. On the other hand, the target sound extraction section 138 calculates a noise sound anticipated component by a BEF (Band Elimination Filter) which interrupts the band which is central to the heart sound X.

Then, the target sound extraction section 138 calculates signals Q and R by averaging each of the output signals from the BPF and the BEF. Afterwards, the target sound extraction section 138 searches for an optimal a by evaluating the signals Q and R. Specifically, the target sound extraction section 138 calculates an optimal a so that the signal Q is maximized and the signal R is minimized, while performing a sequence control to successively change α.

Note that there are cases where the α which maximizes the signal Q and the α which minimizes the signal R do not match. In this case, for example, a weighting is performed on each α, and an optimal α is calculated. Further, since there is the possibility that the optimal α changes with the passing of time, the target sound extraction section 138 may calculate an optimal α again after a prescribed amount of time after calculating the optimal α.

Further, while in the above described example subtraction of the body cavity external signals by the auscultation apparatus 1 and the body cavity internal signals by the capsule 2A is performed on a time axis, the present disclosure is not limited to such an example. For example, this subtraction may be executed as an SS (Spectrum Subtraction) on a frequency axis. Specifically, the target sound extraction section 138 may extract the heart sound X by a difference on a frequency axis between signals in which the heart sound X is predominant (body cavity internal signals or body cavity external signals) and signals in which the noise N is predominant.

(Supplementation)

Note that, while in the above described example the two way reference type NR section 134 combines the body cavity internal signals to which signal processes are performed in the capsule 2, with the body cavity external signals to which similar signal processes are performed, the present disclosure is not limited to such an example. For example, the capsule 2 may transmit, to the auscultation apparatus 1, the body cavity internal signals to which signal processes are not performed, or only minimal signal processes such as band restriction are performed. Also, upon performing signal processes via the NR section 132 and the D-range control processing section 133 or the like, the auscultation apparatus 1 may combine the body cavity internal signals received from the capsule 2 with body cavity external signals by the two way reference type NR section 134.

[2-1-4. Effects]

As described above, it is possible for the sound collection system according to the present embodiment to more clearly acquire target sounds originating at a target part inside the body cavity, by combining body sounds collected from inside the body cavity and body sounds collected from outside of the body cavity. Further, since the distance up to the target part, the state of the surroundings, the sound collection direction and the like are different for the sound collection section 12 outside of the body cavity and the sound collection section 20 inside the body cavity, the two way reference type NR section 134 can extract target sounds with a good S/N ratio, by a combination of sounds with a high degree of independence.

Further, since the capsule 2 collects body sounds from inside the body cavity, the capsule 2 can acquire body sounds more clearly than a general stethoscope which collects body sounds from outside of the body cavity by placing the sound collection section on the body surface. Therefore, the sound collection system according to the present embodiment can extract target sounds more clearly, when compared to a comparative example which extracts target sounds by combining a plurality of body sounds collected from outside the body cavity.

In this way, according to the present embodiment, it becomes possible for early detection or the like of an abnormal part of the heart, lungs or digestive tract, by more clearly acquiring body sounds used for diagnosis of abnormal sounds such as arrhythmia, heart murmurs, wheezing, or bowel sounds, and medical technology will be innovatively advanced.

Further, the auscultation apparatus 1 has a form similar to that of a general stethoscope which an observer of body sounds (a doctor) is familiar with. Therefore, it is possible for an observer to instinctively use the auscultation apparatus 1.

### 2-2. The Second Embodiment

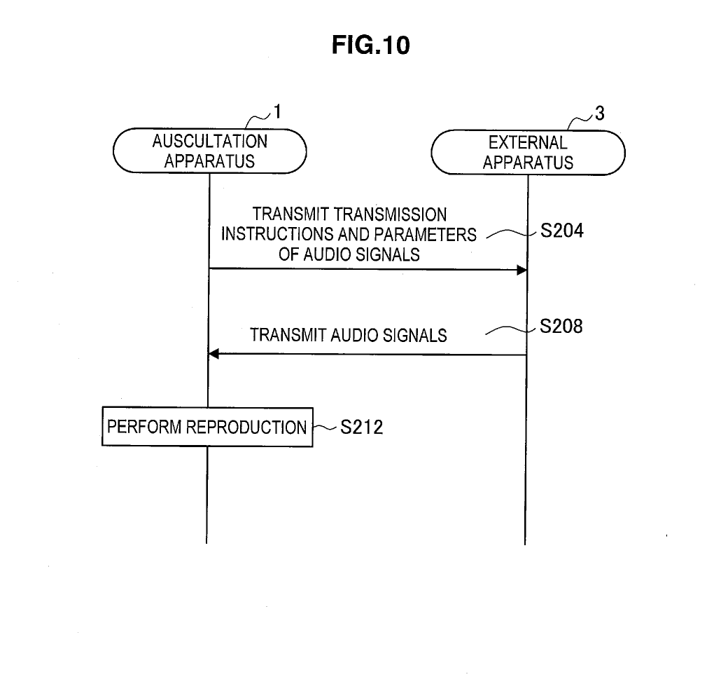

According to the present embodiment, it is possible for the auscultation apparatus 1 to reproduce sounds previously recorded by the external apparatus 3. Since the configuration of the present embodiment follows that described in [2-1-1. Configuration of the capsule 2] and [2-1-2. Configuration of the auscultation apparatus 1], a description of it will be omitted here. Hereinafter, the operations of the present embodiment will be described with reference to FIG. 10.

[2-2-1. Operations]

FIG. 10 is a sequence diagram which shows the operation processes of a sound collection system according to a second embodiment of the present disclosure.

First, in step S204, the transmission section 142 transmits transmission instructions and parameters of the audio signals to the external apparatus 3. The parameters here are information which specifies the target part, position information of the target part, and information such as the previous times to be reproduced.

To continue, in step S208, the external apparatus 3 transmits the audio signals and parameters to the auscultation apparatus 1. The audio signals here are target signals previously received from the auscultation apparatus 1, body cavity external signals, body cavity internal signals, or a combination of these. Further, the parameters are information such as the position and time of the auscultation apparatus 1 and the capsule 2 at the time when the auscultation apparatus 1 previously received these audio signals.

Finally, in step S212, the auscultation apparatus 1 reproduces the audio signals received from the external apparatus 3. In this case, reproduction may be performed without performing any signal processes on the received audio signals. Also, upon performing the above described signal processes for the received body cavity external signals and body cavity internal signals, the auscultation apparatus 1 may reproduce the audio signals as target sounds, based on the received parameters from the two way reference type NR section 134.

[2-2-2. Effects]

According to the present embodiment as described above, the auscultation apparatus 1 can reproduce sounds previously recorded by the external apparatus 3. Accordingly, an observer can perform a more suitable diagnosis by repeatedly listening to the body sounds. Further, the observer of the body sounds can perform a diagnosis by comparing the previous body sounds with the current body sounds.

Further, the auscultation apparatus 1 can receive and reproduce not only the target signals, but also body cavity internal signals and body cavity internal signals to which signal processes are not performed by the two way reference type NR section 134. Therefore, it may not be necessary for the auscultation apparatus 1 and the capsule 2 to repeatedly perform sound collection, even in the case where body sounds to which signal processes are not performed become necessary at a later time for cause investigation of pathology.

### 3. CONCLUSION

It should be understood by those skilled in the art that various modifications, combinations, sub-combinations and alterations may occur depending on design requirements and other factors insofar as they are within the scope of the appended claims or the equivalents thereof.

For example, while in the above described embodiments an internal organ such as the heart, lungs or digestive tract is set as a target part, and the blood flow sound is set as noise, the present disclosure is not limited to such an example. For example, the auscultation apparatus 1 and the capsule 2 may set a blood vessel as a target part, may set sounds originating from internal organs such as the heart, lungs and digestive tract as noise, and may extract and generate the blood flow sound.

Additionally, the present technology may also be configured as below:

(1) A signal processing system including:

a first detection section which detects, from outside a body cavity, first audio signals of a prescribed part inside the body cavity;

a second detection section which detects, from inside the body cavity, second audio signals of the prescribed part inside the body cavity; and

a generation section which generates third audio signals based on the first audio signals and the second audio signals.

(2) The signal processing system according to (1),

wherein the second detection section is a capsule type medical apparatus introduced into the body cavity.

(3) The signal processing system according to (1),

wherein the second detection section is an endoscope type medical apparatus introduced into the body cavity.

(4) The signal processing system according to any one of (1) to (3), further including:

a plurality of the second detection sections.

(5) The signal processing system according to any one of (1) to (4),

wherein the generation section generates the third audio signals upon correcting a time difference between the first audio signals and the second audio signals based on a distance between the first detection section and the prescribed part, and a distance between the second detection section and the prescribed part.

(6) The signal processing system according to (5),

wherein the generation section generates the third audio signals by separating audio signals originating at the prescribed part from the first audio signals and the second audio signals in which the time difference is corrected.

(7) The signal processing system according to any one of (1) to (6), further including:

an encoder section which encodes the third audio signals; and

a transmission section which transmits the third audio signals encoded by the encoder section to an external apparatus.

(8) The signal processing system according to (7), further including:

a reception section which receives, from the external apparatus, third audio signals previously received and accumulated by the external apparatus in accordance with a position of the first detection section.

(9) The signal processing system according to (8), further including:

a reproduction section which reproduces the third audio signals generated by the generation section or the third audio signals received by the reception section.

(10) A signal processing apparatus including:

a first detection section which detects, from outside a body cavity, first audio signals of a prescribed part inside the body cavity; and

a generation section which generates third audio signals based on the first audio signals, and second audio signals of the prescribed part inside the body cavity detected by a second detection section which detects, from inside the body cavity, the second audio signals.

(11) The signal processing apparatus according to (10),

wherein the signal processing apparatus is an electronic auscultation apparatus, and

wherein the second detection section is a capsule type medical apparatus introduced into the body cavity.

(12) The signal processing apparatus according to (10),

wherein the signal processing apparatus is an electronic auscultation apparatus, and

wherein the second detection section is an endoscope type medical apparatus introduced into the body cavity.

(13) The signal processing apparatus according to any one of (10) to (12),

wherein the generation section generates the third audio signals based on the first audio signals and a plurality of the second audio signals detected by a plurality of the second detection sections.

(14) The signal processing apparatus according to any one of (10) to (13),

wherein the generation section generates the third audio signals upon correcting a time difference between the first audio signals and the second audio signals based on a distance between the first detection section and the prescribed part, and a distance between the second detection section and the prescribed part.

(15) A non-transitory computer-readable storage medium having a program stored thereon, the program causing a computer to execute:

detecting, from outside a body cavity, first audio signals of a prescribed part inside the body cavity;

detecting, from inside the body cavity, second audio signals of the prescribed part inside the body cavity; and

generating third audio signals based on the first audio signals and the second audio signals.

(16) A non-transitory computer-readable storage medium having a program stored thereon, the program causing a computer to execute:

detecting, from outside a body cavity, first audio signals of a prescribed part inside the body cavity; and

generating third audio signals based on the first audio signals, and second audio signals of the prescribed part inside the body cavity detected from inside the body cavity.

The present disclosure contains subject matter related to that disclosed in Japanese Priority Patent Application JP 2012-188800 filed in the Japan Patent Office on Aug. 29, 2012, the entire content of which is hereby incorporated by reference.

What is claimed is:

1. A signal processing system comprising:
a first detection section which detects, from outside a body cavity, first audio signals of a prescribed part inside the body cavity;
a second detection section which detects, from inside the body cavity, second audio signals of the prescribed part inside the body cavity; and
a generation section which generates third audio signals based on the first audio signals and the second audio signals.

2. The signal processing system according to claim 1,
wherein the second detection section is a capsule type medical apparatus introduced into the body cavity.

3. The signal processing system according to claim 1,
wherein the second detection section is an endoscope type medical apparatus introduced into the body cavity.

4. The signal processing system according to claim 1, further comprising:
a plurality of the second detection sections.

5. The signal processing system according to claim 1,
wherein the generation section generates the third audio signals upon correcting a time difference between the first audio signals and the second audio signals based on a distance between the first detection section and the prescribed part, and a distance between the second detection section and the prescribed part.

6. The signal processing system according to claim 5,
wherein the generation section generates the third audio signals by separating audio signals originating at the prescribed part from the first audio signals and the second audio signals in which the time difference is corrected.

7. The signal processing system according to claim 1, further comprising:
an encoder section which encodes the third audio signals; and
a transmission section which transmits the third audio signals encoded by the encoder section to an external apparatus.

8. The signal processing system according to claim 7, further comprising:
a reception section which receives, from the external apparatus, third audio signals previously received and accumulated by the external apparatus in accordance with a position of the first detection section.

9. The signal processing system according to claim 8, further comprising:
a reproduction section which reproduces the third audio signals generated by the generation section or the third audio signals received by the reception section.

10. A signal processing apparatus comprising:
a first detection section which detects, from outside a body cavity, first audio signals of a prescribed part inside the body cavity; and
a generation section which generates third audio signals based on the first audio signals, and second audio signals of the prescribed part inside the body cavity detected by a second detection section which detects, from inside the body cavity, the second audio signals.

11. The signal processing apparatus according to claim 10,
wherein the signal processing apparatus is an electronic auscultation apparatus, and
wherein the second detection section is a capsule type medical apparatus introduced into the body cavity.

12. The signal processing apparatus according to claim 10,
wherein the signal processing apparatus is an electronic auscultation apparatus, and
wherein the second detection section is an endoscope type medical apparatus introduced into the body cavity.

13. The signal processing apparatus according to claim 10,
wherein the generation section generates the third audio signals based on the first audio signals and a plurality of the second audio signals detected by a plurality of the second detection sections.

14. The signal processing apparatus according to claim 10,
wherein the generation section generates the third audio signals upon correcting a time difference between the first audio signals and the second audio signals based on a distance between the first detection section and the prescribed part, and a distance between the second detection section and the prescribed part.

15. A non-transitory computer-readable storage medium having a program stored thereon, the program causing a computer to execute:
detecting, from outside a body cavity, first audio signals of a prescribed part inside the body cavity;
detecting, from inside the body cavity, second audio signals of the prescribed part inside the body cavity; and
generating third audio signals based on the first audio signals and the second audio signals.

16. A non-transitory computer-readable storage medium having a program stored thereon, the program causing a computer to execute:
detecting, from outside a body cavity, first audio signals of a prescribed part inside the body cavity; and
generating third audio signals based on the first audio signals, and second audio signals of the prescribed part inside the body cavity detected from inside the body cavity.
